(19) United States
(12) Patent Application Publication
(10) Pub. No.: US 2024/0131022 A1
(43) Pub. Date: Apr. 25, 2024

(54) OPIOID OVERDOSE REVERSAL MIXTURES

(71) Applicants: John ABERNETHY, Gainesville, FL (US); Georgiy NIKONOV, Alachua, FL (US); Michael VORONKOV, Chelsea, MA (US)

(72) Inventors: John ABERNETHY, Gainesville, FL (US); Georgiy NIKONOV, Alachua, FL (US); Michael VORONKOV, Chelsea, MA (US)

(21) Appl. No.: 18/334,528

(22) Filed: Jun. 14, 2023

Related U.S. Application Data
(63) Continuation-in-part of application No. 17/803,164, filed on Mar. 11, 2022.

(51) Int. Cl. A61K 31/485; A61K 9/00; A61P 25/36

(52) U.S. Cl. CPC A61K 31/485; A61K 9/0043; A61P 25/36

(57) ABSTRACT

The subject invention pertains to compositions comprising an opioid receptor antagonist and an opioid receptor agonist and methods of inhibiting an opioid overdose in a subject. The opioid receptor antagonist can be naloxone or a derivative thereof, while the opioid receptor agonist can be nalbuphine or a derivative thereof. The composition can be administered intranasally, intravenously and by autoinjector. The reversal time can be about three minutes.

### CROSS-REFERENCE TO RELATED APPLICATION

This application is the continuation-in-part of U.S. application Ser. No. 17/803,164, filed Mar. 11, 2022, the disclosure of which is incorporated herein by reference in its entirety.

### FIELD OF THE INVENTION

The present invention relates to opioid derived compositions, used in reversing opioid overdose.

### BACKGROUND OF THE INVENTION

Naloxone has been a gold standard for reversing an opioid overdose.¹ Since overdose is an adverse effect of an opioid receptor activation, the pharmacological role of naloxone as an antagonist is to outcompete other opioids for the receptor. The rising death toll from overdoses induced by non-medical fentanyl, a highly potent opioid, however requires expeditious delivery of naloxone at higher/repeated doses pushing the limits of the intervention safety². Yet, the administration of naloxone to overdose victims has its own liabilities.

Naloxone, sold under the brand name Narcan® (Emergency Devices Inc., Plymouth Meeting, PA), is a medication used to block the effects of opioids, especially in overdose situations. Naloxone may also be combined with an opioid (in the same pill or compound), to decrease the risk of opioid misuse. For instance, it can be added to the coating for a sustained release opiate compound, to prevent crushing of the sustained release compound, which could lead to an overdose.

When given intravenously, naloxone typically works within two minutes, and when injected into a muscle, it works within five minutes. It may also be sprayed into the nose. The effects of naloxone typically last for about half an hour to an hour. Thus, multiple doses and administration of naloxone may be required, as the duration of action of most opioids is greater than that of naloxone.

Administration of naloxone to opioid-dependent individuals may cause symptoms of opioid withdrawal, such as, for example, restlessness, agitation, nausea, vomiting, increased heart rate and perspiration. To prevent this, small doses of naloxone can be given every few minutes until the desired effect is reached.

In individuals with prior history of heart disease or who take medications that negatively affect the heart, further heart problems have occurred. Naloxone appears to be safe in pregnancy, after having been given to and tested on a limited number of subjects.

Naloxone is a non-selective and competitive opioid receptor antagonist. It works by reversing the depression of the central nervous system and respiratory system caused by opioids.

Naloxone, also known as N-allylnoroxymorphone or as 17-allyl4,5α-epoxy-3,14-dihydroxymorphinan-6-one, is a synthetic morphinan derivative and was derived from oxymorphone (14-hydroxydihydromorphinone), an opioid analgesic. Oxymorphone, in turn, was derived from morphine, an opioid analgesic and naturally occurring constituent of the opium poppy.

Naloxone is a racemic mixture of two enantiomers, (−)-naloxone (levonaloxone) and (+)-naloxone (dextronaloxone), only the former of which is active at opioid receptors. The drug is a highly lipophilic, allowing it to rapidly penetrate the brain and to achieve a far greater brain to serum ratio than that of morphine. Opioid antagonists related to naloxone include cyprodime, nalmefene, nalodeine, naloxol, and naltrexone.

Naloxone precipitates severe withdrawal sickness^{3,4}, a life-threatening condition.⁵ Withdrawal syndrome requires active management of symptoms with additional medications that include pain medications for myalgia and medicines for cardiovascular and gastrointestinal side effects.⁵ Furthermore, it is well documented that in some instances the naloxone induced withdrawal forces patients to self-medicate with street drugs to counter the naloxone effects.⁶ Such practices contribute to the significant rate of post-rescue deaths immediately following hospital discharge.⁷ Moreover, hyperalgesia and lower pain tolerance during the opioid withdrawal are prominently associated with increased relapse rates.⁸

Second of all, naloxone administration is associated with catecholamine release⁹ that is thought to be involved in pulmonary edema¹⁰ and cardiovascular stimulation¹¹, the most prevalent side effects in overdose patients. These may lead to serious adverse events reported for doses above 0.002 mg/kg that raises questions regarding a safe dose in patients susceptible to naloxone induced withdrawal.¹⁰

Despite these limitations, it is generally assumed that higher doses of naloxone are needed to reverse a fentanyl-driven overdose. Higher doses were rationalized through fentanyl competitive affinity for and kinetics of interaction with the opioid receptors that affect the robustness of naloxone-assisted reversals.^{12,13} Therefore, there is an unmet medical need to develop a fentanyl-induced overdose reversal agent that is not only more effective than naloxone, but also has better withdrawal and safety profiles.

### BRIEF SUMMARY OF THE INVENTION

The subject invention relates to a remedial opioid overdose composition comprising a therapeutically effective dose of an opioid receptor antagonist and a therapeutically effective dose of a dual opioid receptor agonist/antagonist. In certain embodiments, the composition can be applied by an injection (e.g., subcutaneous, intravenously, intramuscularly) or, preferably, intranasally to a subject suffering from an opioid overdose. The composition can reverse the opioid overdose in the subject. In one embodiment, the opioid receptor antagonist is naloxone and the opioid receptor agonist/antagonist is nalbuphine. In another embodiment, the overdose composition can reverse an opioid overdose in about three minutes or less.

The present invention also broadly comprises a remedial opioid overdose mixture that comprises: a therapeutically effective dose of an opioid receptor mu antagonist, such as for example, 3-hexadienoate derivative of naloxone (NX90; formula (I)); and, a therapeutically effective dose of an opioid receptor kappa agonists, such as, for example, 3-hexadienoate derivative of nalbuphine (NB33; formula (II)). In preferred embodiments, the composition comprises NX90 and NB33, naloxone and NB33, NX90 and nalbuphine, or naloxone and nalbuphine. In certain embodiments, the opioid overdose mixture can be applied intramuscularly, by an injection (e.g., intravenously), or, preferably, intranasally to an opioid overdose patient. NB33 is a 3-hexadienoate derivative of nalbuphine that converts to the parent drug in a biological matrix. NX90 is a 3-hexadienoate derivative of naloxone that converts to the parent drug in a biological matrix.

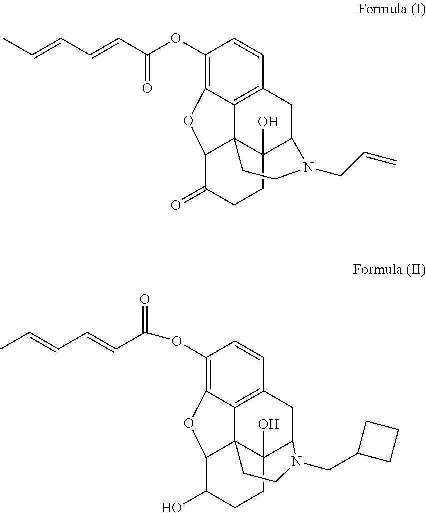

In certain embodiments, the subject compositions and methods can reverse an opioid overdose within or equal to about 3 minutes, about 2.75 minutes, about 2.5 minutes, about 2.25 minutes, about 2 minutes, or less. In certain embodiments, the subject compositions and methods can reverse a fentanyl overdose within or equal to about 3 minutes, about 2.75 minutes, about 2.5 minutes, about 2.25 minutes, about 2 minutes, about 100 seconds, about 90 seconds, about 80 seconds, about 70 seconds, about 60 seconds, about 50 seconds, about 40 seconds, about 30 seconds, or less.

In certain embodiments, the subject compositions and methods can lower the cardiorespiratory risks burden and reduction of withdrawal severity of naloxone in opioid person susceptible to withdrawal.

### BRIEF DESCRIPTION OF THE DRAWINGS

FIGS. 1A-1D. FIG. 1A. Design of the experiment; vitals and reflexes monitored: RR—respiratory rate, HR—heart rate, BT—body temperature, AN—alertness, AT—astasia, CR—corneal reflex, PRT—pinch reflex tail, PRToe—pinch reflex toe, Rref—righting reflex, ST—sternal recumbency. FIG. 1B. Mean time to all reflexes restored at 0.31 and 0.62 μmol/kg doses. FIG. 1C. Mean time to all reflexes restored at 1.24 μmol/kg doses. FIG. 1D. RR AUC (RR×min) for each treated group. Resting RR AUC was calculated by multiplying average resting RR by 60 min.

FIG. 2 Mean heart rate (HR) for the naloxone (1.24 μmol/kg), NX90 (1.24 μmol/kg) or NB33+NX90 (0.31+0.31 μmol/kg and 0.62+0.62 μmol/kg) treated groups (n=5) in fentanyl driven OD model.

FIG. 3 depicts dose rate comparisons of types and quantities of the test substances on OD reversal in subject rats.

FIGS. 4A-4B. FIG. 4A. Design of the experiment; vitals and reflexes monitored: RR—respiratory rate, HR—heart rate, BT—body temperature, AN—alertness, AT—astasia, CR—corneal reflex, PRT—pinch reflex tail, PRToe—pinch reflex toe, Rref—righting reflex, ST—sternal recumbency. T−20 NOC—nociception pretest 20 min prior fentanyl administration, T+40 nociception test 40 min after fentanyl administration. FIG. 4B. Mean time to all reflexes restored.

FIGS. 5A-5D. FIG. 5A. Mean heart rate (RR) for as percent of resting rate for all treated groups. FIG. 5B. Cumulative measure of respiratory activity for all treated groups (*p<0.05). FIG. 5C. Mean heart rate (HR) for as percent of resting rate for all treated groups. FIG. 5D. Cumulative measure of respiratory activity for all treated groups (*p<0.05; **p<0.01; ***p<0.005).

FIGS. 6A-6B. FIG. 6A. Analgesia as percent of maximum possible effect for all treated groups. FIG. 6B. Rank order of OD reversal (mean time to all reflexes restored), HR AUC, RR AUC and analgesia for all interventions.

### DETAILED DESCRIPTION OF EMBODIMENTS OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X 6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a drug. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as prevent or treat an opioid overdose. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the state of the subject being treated, such as, for example, whether or not the subject is conscious, has respiratory suppression, has a lower body temperature, has a lower heart rate, or has a loss of reflexes (e.g., corneal). In some embodiments of the invention, the method comprises administration of the composition sequentially, including, but not limited to, 2 times sequentially, 3 times sequentially, 4 times sequentially, or more. In certain embodiments, the 2^{nd}, 3^{rd} or 4^{th} sequential administration can be performed by injection or infusion.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, an “overdose reversal” or “opioid overdose reversal” refers to the process by which a compound binds to the opioid receptors or otherwise blocks the opioids receptors, partially or completely, in a subject, which results in reversal of life threatening effects such as, for example, respiratory depression.

“Pharmaceutically acceptable” refers to a substance having pharmacological properties consistent with medical use.

“Pharmaceutically acceptable salt” refers to a compound of the invention that is pharmaceutically acceptable and that possesses the desired pharmacological activity of the parent compound. In particular, such salts that are non-toxic may be inorganic or organic acid addition salts and base addition salts.

“Pharmaceutically acceptable vehicle” refers to a diluent, adjuvant, excipient, propellent or carrier with which a compound of the invention is administered. A “pharmaceutically acceptable vehicle” refers to a substance that is non-toxic, biologically tolerable, and otherwise biologically suitable for administration to a subject, such as an inert substance, added to a pharmacological composition or otherwise used to facilitate administration of an agent and that is compatible therewith. Examples of vehicles include but are not limited to calcium carbonate, calcium phosphate, various sugars and types of starch, cellulose derivatives, gelatin, vegetable oils, and polyethylene glycols.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Opioid Reversal Compositions

The subject invention pertains to compositions comprising an opioid receptor mu antagonist or a pharmaceutically acceptable salt or derivative thereof, an opioid receptor kappa agonist or a pharmaceutically acceptable salt or derivative thereof, or a combination thereof and methods of treatment of an opioid overdose or symptoms thereof comprising administering the composition to a subject in need thereof.

In certain embodiments, the opioid receptor mu antagonist is naloxone, naltrexone, samidorphan, nalorphine, diprenorphine, levallorphan, nalmefene or a pharmaceutically acceptable salt or a derivative thereof and the opioid receptor kappa agonist is nalbuphine butorphanol, pentazocine, or a pharmaceutically acceptable salt or a derivative thereof. In certain embodiments, the derivative of naloxone is a 3-hexadienoate derivative of naloxone (NX90) and the derivative of nalbuphine is a 3-hexadienoate derivative of nalbuphine (NB33). In certain embodiments, other hexadienoate derivatives of naloxone can be used, such as, for example, naloxone-3-(5-methyl)hexadienoate. In certain embodiments, other hexadienoate derivatives of nalbuphine can be used, such as, for example, nalbuphino-3-(5-methyl)hexadienoate; 3-(cyclobutylmethyl)-9-(((2E,4E)-hexa-2,4-dienoyl)oxy)-4a,7-dihydroxy-2,3,4,4a,5,6,7,7a-octahydro-1H-4,12-methanobenzofuro[3,2-e]isoquinolin-3-ium chloride; or 7-acetoxy-3-(cyclobutylmethyl)-4a-hydroxy-2,3,4,4a,5,6,7,7a-octahydro-1H-4,12-methanobenzofuro[3,2-e]isoquinolin-9-yl (2E,4E)-hexa-2,4-dienoate. In certain embodiments, the composition can further comprise buprenorphine.

In certain embodiments, the therapeutically effective dose of the opioid receptor mu antagonist can be about 0.005 mg/kg to about 1 mg/kg, about 0.05 mg/kg about 0.2 mg/kg, about 0.1 mg/kg to about 0.2 mg/kg, about 0.3 mg/kg to about 0.8 mg/kg or about 0.0038 mg/kg to about 0.0145 mg/kg body weight of a subject.

In certain embodiments, the therapeutically effective dose of the opioid receptor kappa agonist can be about 0.001 mg/kg to about 4 mg/kg, 0.001 mg/kg to about 2.5 mg/kg, 0.001 mg/kg to about 2.25 mg/kg, 0.001 mg/kg to about 2.0 mg/kg, 0.001 mg/kg to about 1.75 mg/kg, 0.001 mg/kg to about 1.5 mg/kg, 0.001 mg/kg to about 1.25 mg/kg, 0.001 mg/kg to about 1.0 mg/kg, about 0.0013 mg/kg to about 0.8 mg/kg, about 0.005 mg/kg to about 1 mg/kg, about 0.05 mg/kg about 0.2 mg/kg, about 0.1 mg/kg to about 0.2 mg/kg, about 0.3 mg/kg to about 0.8 mg/kg, or about 0.037 mg/kg to about 0.073 mg/kg about body weight of a subject.

In certain embodiments, the composition can comprise a therapeutically effective dose of the opioid receptor mu antagonist at the same amount to the therapeutically effective dose of the opioid receptor kappa agonist. In certain embodiments, the therapeutically effective dose of the opioid receptor mu antagonist and the opioid receptor kappa agonist is 0.001 mg/kg to about 2.5 mg/kg, about 0.013 mg/kg to about 0.8 mg/kg or about 0.05 mg/kg or about 0.1 mg/kg body weight of a subject.

In alternative embodiments, the composition can comprise a therapeutically effective dose of the opioid receptor mu antagonist at a ratio of about 4:1 to about 1:8 relative to the therapeutically effective dose of the opioid receptor kappa agonist. In certain embodiments, the therapeutically effective dose of the opioid receptor mu antagonist is at a ratio of about 4:1, about 1:4, about 1:5, or about 1:8 relative to the therapeutically effective amount of the opioid receptor kappa agonist. In preferred embodiments, the therapeutically effective dose of the opioid receptor mu antagonist is about 0.05 mg/kg to about 0.1 mg/kg and the therapeutically effective dose of the opioid receptor kappa agonist is about 0.013 mg/kg to about 0.8 mg/kg, about 0.025 mg/kg to about 0.5 mg/kg or about 0.025 to about 0.4 mg/kg body weight of the subject.

In certain embodiments, the opioid receptor mu antagonist and the opioid receptor kappa agonist can be administered concurrently or in series. In certain embodiments, the opioid receptor mu antagonist and the opioid receptor kappa agonist can be administered within about 1 sec, about 2 sec, about 5 sec, about 10 sec, about 30 sec, about 60 sec, about 2 min, about 5 min, or about 10 min. In certain embodiments, the opioid receptor mu antagonist can be administered before the opioid receptor kappa agonist. In certain embodiments, the opioid receptor mu antagonist can be administered after the opioid receptor kappa agonist.

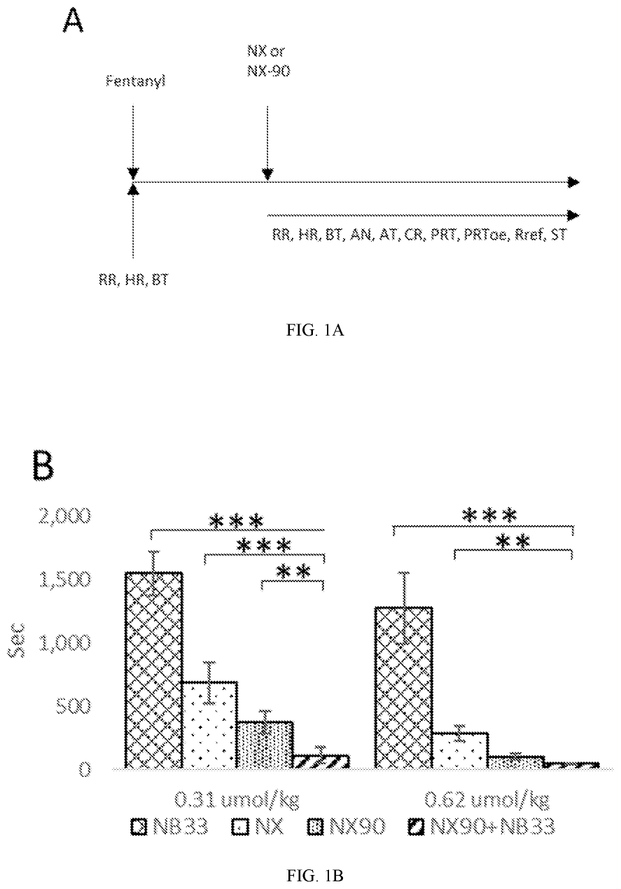

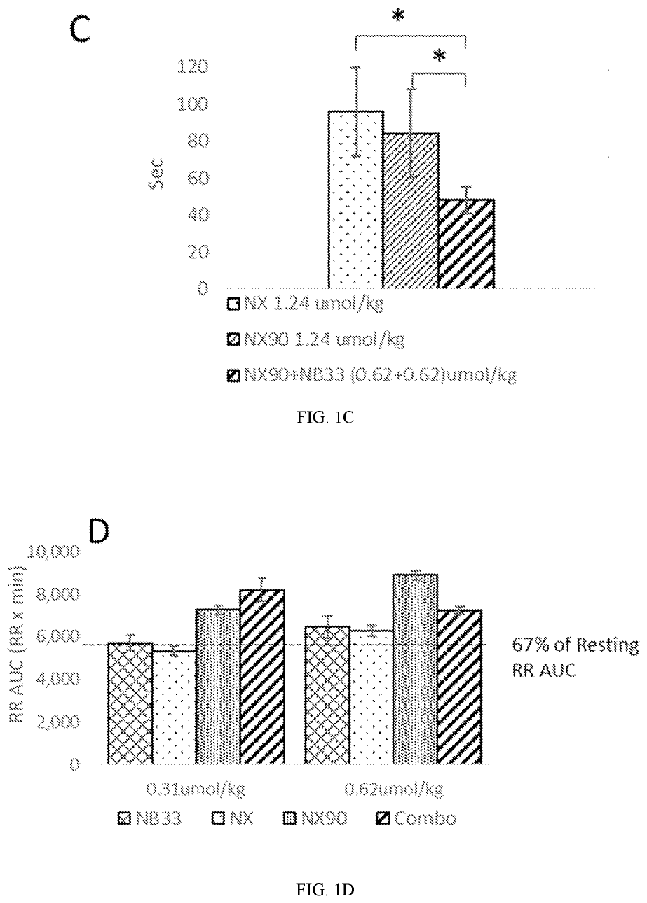

In certain embodiments, co-administration of a mu-antagonist (e.g., NX90) with a kappa agonist (e.g., NB33) can produce a synergistic effect in reversal of an opioid overdose (e.g., fentanyl) that can be characterized by shorter total recovery times and a reliable reversal an opioid overdose in each subject. The efficacy of reversing a non-heroin overdose, speed of recovery, and more universal response of subjects to intranasal administration is crucial to preventing overdose in street settings by the first responders, police and opiate users and offers significant advantage over current therapeutic options. The administration of naloxone alone cannot reverse an overdose in fewer than about 90 seconds, irrespective of the dose of naloxone. In preferred embodiments, the subject compositions can reverse an overdose in less than about 90 seconds, about 80 seconds, about 70 seconds, about 60 seconds, or about 50 seconds, optionally at a half of the dose of naloxone found in commercially available naloxone pharmaceuticals, such as, for example, Narcan® (Emergent Devices Inc. Plymouth Meeting, PA) (FIG. 1C).

In certain embodiments, co-administration of a mu-antagonist (e.g., NX90) with a kappa agonist (e.g., NB33) to a subject suffering from an overdose can mitigate opioid overdose non-respiratory sides effects. In some embodiments, the mitigated non-respiratory side effects are associated with catecholamine release (e.g., cardiovascular stimulation and pulmonary edema). In other embodiments, the mitigated non-respiratory side effects are associated with the pain aspect of acute withdrawal and can further inhibit post-discharge risky behavior⁷ (e.g., people who were rescued from an overdose immediately can seek opioids to mitigate severity of naloxone-induced withdrawal and can die as a result). In certain embodiments, the subject compositions may be utilized for the treatment of side effects of opioid agonists including, for example, pruritus, respiratory suppression, lower body temperature, lower heart rate, loss of reflexes (e.g., corneal), and overdose.

In certain embodiments, the subject compositions can be used to reverse an opioid overdose in a subject. In certain embodiments, the opioid is oxycodone, oxymorphone, hydrocodone, hydromorphone, fentanyl, morphine, codeine, methadone, tramadol, buprenorphine, heroin, or any combination thereof. In certain embodiments, the opioid overdose may also be caused by administration of an opioid with a sedative (e.g., xylazine, nitazines, propofol, ketamine, or thiopental), muscle relaxant, or anesthetic agent. In preferred embodiments, the subject compositions and methods can reverse an opioid overdose within or equal to about 90 seconds, about 80 seconds about 70 seconds, about 60 seconds, about 50 seconds, about 45 seconds, about 30 seconds, or less time. In certain embodiments, the subject compositions and methods can reverse a fentanyl overdose within or equal to about 90 seconds, about 80 seconds about 70 seconds, about 60 seconds, about 50 seconds, about 45 seconds, about 30 seconds, or less time.

In certain embodiments, the addition of the kappa agonist to the mu antagonist mitigates side effects and the cardiorespiratory risks burden of the use of the mu antagonist, normalizes the heart rate of the subject, and lowers withdrawal severity of the subject.

Salts of Receptor Mu Antagonists and Opioid Receptor Kappa Agonists of the Invention

In some embodiments the subject invention provides salts of the receptor mu antagonists and opioid receptor kappa agonists described herein. The salts can be a salt with an inorganic acid, such as hydrochloric acid, hydrobromic acid, perchloric acid, nitric acid, thiocyanic acid, sulfuric acid, and phosphoric acid; an organic acid, such as trifluoroacetic acid (TFA), formic acid, acetic acid, propionic acid, glycolic acid, lactic acid, pyruvic acid, oxalic acid, malonic acid, succinic acid, maleic acid, and fumaric acid.

Further salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and when the receptor mu antagonists and opioid receptor kappa agonists contain a basic functionality, salts of non-toxic organic or inorganic acids, such as hydrochloride, hydrobromide, tartrate, mesylate, acetate, maleate, oxalate and the like.

Certain embodiments provide amorphous forms of salts of the receptor mu antagonists and opioid receptor kappa agonists disclosed herein. Such amorphous forms are advantageous for nasal delivery.

### Methods of Treatment

Administration of composition of the subject invention can be carried out in the form of an spray, mist, or liquid formulation containing a therapeutically effective amount of the active ingredients (opioid receptor mu antagonists and opioid receptor kappa agonists). In preferred embodiments, the delivery mode of subject compounds is nasally. Administration is not limited to nasal delivery and includes intravascular (e.g., intravenous), intraperitoneal, intramuscular, or another means known in the pharmaceutical art for administration of active pharmaceutical ingredients.

Therapeutic or prophylactic application of the opioid receptor mu antagonists and opioid receptor kappa agonists and compositions containing thereof, can be accomplished by any suitable therapeutic or prophylactic method and technique presently or prospectively known to those skilled in the art. The opioid receptor mu antagonists and opioid receptor kappa agonists can be administered by any suitable route known in the art including, for example, oral, intramuscular, intraspinal, intracranial, nasal, parenteral, subcutaneous, or intravascular (e.g., intravenous) routes of administration. Administration of the opioid receptor mu antagonists and opioid receptor kappa agonists of the invention can be continuous or at distinct intervals as can be readily determined by a person skilled in the art.

In some embodiments, an amount of opioid receptor mu antagonists and opioid receptor kappa agonists can be administered 1, 2, 3, 4, or more times per day. Optionally, the treatment regimen can include a loading dose, with one or more daily maintenance doses. For example, in some embodiments, an initial loading dose in the range 0.005 mg/kg body weight to about 0.5 mg/kg body weight of a subject, as needed to inhibit an overdose in a subject or at specific time interval, such as for example, every 12 hours for 1, 2, 3, 4, 5, 6, 7, or more days.

Opioid receptor mu antagonists and opioid receptor kappa agonists and compositions comprising said compounds can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the opioid receptor mu antagonist and the opioid receptor kappa agonist is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject compounds include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the subject compounds can also comprise suspension agents, protectants, lubricants, buffers, preservatives, and stabilizers. To provide for the administration of such dosages for the desired therapeutic treatment, pharmaceutical compositions of the invention will advantageously comprise between about 0.001 mg/mL to about 10 mg/mL, about 0.01 mg/mL to about 5 mg/mL, about 0.02 mg/mL to about 1 mg/mL, or about 0.4 mg/mL of the total of one or more of the subject compounds based on the weight of the total composition including carrier or diluent.

The subject invention also concerns a packaged dosage formulation comprising in one or more packages, packets, or containers at least one opioid receptor mu antagonist and opioid receptor kappa agonist and/or composition of the subject invention formulated in a pharmaceutically acceptable dosage. The package can contain discrete quantities of the dosage formulation, such as sprays, liquids, capsules, lozenge, and powders. In some embodiments, the opioid receptor mu antagonist and opioid receptor kappa agonist can be administered 1, 2, 3, 4, 5, 6, 7, 8, 9, 10 or more times per day, for 2, 3, 4, 5, 6, 7 or more days.

The subject invention also concerns kits comprising in one or more containers an opioid receptor mu antagonist and an opioid receptor kappa agonist. A kit of the invention can also comprise one or more compounds, biological molecules, or drugs. In one embodiment, a kit of the invention comprises an opioid receptor mu antagonist and an opioid receptor kappa agonist.

Optionally, the methods further comprise, prior to administering the opioid receptor mu antagonist and opioid receptor kappa agonist to the subject, identifying the subject as having an overdose, preferably an opioid overdose, or not having an opioid overdose. If the subject is identified as having an opioid overdose, the opioid receptor mu antagonist and opioid receptor kappa agonist can be administered to the human subject as therapy. If the human subject is identified as not having an opioid overdose, the opioid receptor mu antagonist and opioid receptor kappa agonist can be withheld, or the opioid receptor mu antagonist and opioid receptor kappa agonist can be administered as prophylaxis, or an alternative agent can be given.

The subject may be any age or gender. In some cases, the subject may be an infant or older adult. In some embodiments, the subject is 10 years of age or older. In some embodiments, the subject is 20 years of age or older. In some embodiments, the subject is 30 years of age or older. In some embodiments, the subject is 40 years of age or older. In some embodiments, the subject is 55 years of age or older. In some embodiments, the subject is 60 years of age or older.

The invention further provides kits, including an opioid receptor mu antagonist and an opioid receptor kappa agonist and pharmaceutical formulations thereof, packaged into suitable packaging material, optionally in combination with instructions for using the kit components, e.g., instructions for performing a method of the invention. In one embodiment, a kit includes an amount of an opioid receptor mu antagonist and an opioid receptor kappa agonist and instructions for administering the compounds to a subject in need of treatment on a label or packaging insert. In further embodiments, a kit includes an article of manufacture, for delivering the subject compounds into a subject locally, regionally or systemically, for example.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating an opioid overdose. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to treat an opioid overdose. Instructions may additionally include appropriate administration route, dosage information, indications of a satisfactory clinical endpoint or any adverse symptoms that may occur, storage information, expiration date, or any information required by regulatory agencies such as the Food and Drug Administration, Medicines and Healthcare products Regulatory Agency, or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video and additionally be included on a computer readable medium, such as electrical storage media such as RAM and ROM and hybrids of these such as magnetic/optical storage media.

Kits can additionally include a buffering agent, a preservative, or an agent for stabilizing the opioid receptor mu antagonist and opioid receptor kappa agonist. Each component of the kit can be enclosed within an individual container or in a mixture and all of the various containers can be within single or multiple packages.

In all cases it is understood that the above-described examples and compounds are merely illustrative of the many possible specific embodiments which represent applications of the present invention. Numerous and varied other arrangements can be readily devised in accordance with the principles of the present invention without departing from the spirit and the scope of the invention.

In another embodiment, the present invention could also be practiced with other kappa-agonists such as, but not limited to, Nalbuphine, Pentazocine, Butorphanol or related compounds or mu-antagonists such as, but not limited to Naltrexone, Naloxone or related compounds.

In a further one or more embodiments, the present invention is can be practiced with combination of mu-antagonists with kappa-agonists as well as their esters in pharmaceutically acceptable salts to reverse overdose when given intravenously, intranasally, transdermally, sublingually, rectally, topically, intramuscularly, subcutaneously or via inhalation.

### Materials and Methods

### Chemicals

Fentanyl (Fentanyl-Richter, 5 mL vial, 50 μg/mL) and naloxone (Forvel-Medochemie, 1 mL vial, 0.4 mg/mL) were purchased from commercial sources. Nalbuphine was used as the active substance, dissolved in 0.9% saline solution in a concentration of 0.8 mg/mL. NX90 and NB33 were synthesized as previously reported^{14,19}.

### Fentanyl-Induced Overdose Model

Selection of optimal fentanyl dose. We used a single ascending dose study in 15 rats (Rattus norvegicus, Wistar; both genders) to determine the optimal fentanyl (Fentanyl-Richter, 5 mL vial, 50 μg/mL) dose. Starting from ED50 for rats (mentioned in the SPC of the product) of 0.013 mg/kg. Five doses (0.013; 0.026; 0.052; 0.104 and 0.130 mg/kg) were tested on separate groups (1M+2F or 2M+1F) of rats. Monitoring 10 parameters, we selected 0.130 mg/kg of fentanyl for the study. To avoid animal's death from cardiopulmonary arrest, naloxone (Forvel-Medochemie, 1 mL vial, 0.4 mg/mL) was administered immediately upon reaching an overdose.

Main study. In this study, were used a total of 20 rats (Rattus norvegicus, Wistar; both genders) obtained from the laboratory animal facility—Centre for Experimental Medicine, University of Medicine and Pharmacy, Cluj-Napoca, Romania. The age of the rats ranged 5-6 months, and the body weight ranged 201-415 grams. Rats were acclimated for 1 week in a climate-controlled room maintained at 22° C. with approximately 60% relative humidity. Lighting was on a 12-hr light/dark cycle, with food and water available ad libitum. Rats were randomized to 4 groups by body weight, each group consisting of 3M+2F or 2M+3F. Fentanyl was administered in the dose of 0.130 mg/kg, intramuscularly and nalbuphine (active substance in powder form dissolved in sterile saline solution 0.9% in a concentration of 0.8 mg/mL) were administered intranasally. Individual clinical monitoring (EDAM—IM8 VET system) and evaluation was performed at all stages of the study before (ATp) and after (PTp) the administration of pharmacological substances.

Following fentanyl administration (PTp), rats were monitored first 2 minutes continuously and then at 2-minute intervals within the first 10 minutes and then at 10-minute intervals, up to 60 minutes. Fentanyl overdose was considered to have occurred at the time when all reflexes disappeared completely, accompanied by a significant reduction in respiratory and heart rates. At that time, depending on the group tested, NB or its combination with NX was administered intranasally as aqueous solutions of their hydrochlorides, marking the beginning of a time interval required for complete recovery of the 10 monitored reflexes.

### Nociception Evaluation.

Determination of the antinociceptive effect was done using tail flick test (TFT) with ethanol at −25° C., described by Wang et al. (1995) and Chu et al. (2003). The Neslab RTE 7 apparatus with an adjustable temperature between −25° C. and +150° C. (±0.01° C.) and 70% ethanol solution was used. The rats were placed in a transparent containment tube, only the tail being free. Half of the tail end was immersed in the 70% cold ethanol solution. The nociceptive threshold was taken as the latency until the rat flicked its tail from the bath. The time from immersion to tail flick was measured to the nearest hundredths of a second with a laboratory timer. To prevent tissue damage, a predetermined cutoff time of 40 seconds was used. No rats had frostbite or tail color change during the experiment. TFT was performed 20 minutes before Fentanyl administration and again after 40 minutes. The maximum possible analgesia (% MPA) was calculated based on the formula: % MPA=[(Test−Pretest)/(Cutoff−Pretest)]×100%.

### Statistical Analysis

Statistical analysis was performed using IBM SPSS Statistics for Windows, version 27.0 (IBMCorp, Armonk, NY, USA). One-way ANOVA with Bonferroni post hoc test was used to compare differences of mean times to all reflexes restored since baseline, across three treatment groups at each time interval. ANOVA was also used to compare HR changes in all treatment groups. The statistical significance level was set at a<0.05.

### Example 1—Efficacy Assessment of Combinations (e.g., NX90+N33, NX+NB) Vs Naloxone in Fentanyl Driven Overdose Animals

Clinical monitoring, respectively clinical evaluation was performed in all stages of the study before and after the administration of pharmacological substances: for Fentanyl (FT), and test articles (e.g., Naloxone (NX), Nalbuphine (NB), NX90, NB33) before treatment and one hour after treatment (2 minute intervals within the first 10 minutes and then at 10-minute interval).

The rats from each group were treated with Fentanyl, IM (intramuscular), at the set dose (0.130 mg/kg). After sedation/analgesia were installed, a test article was administered IN (intranasally) at the specific dose. The doses of test articles used ranged from 0.31 μmol/kg to 1.24 μmol/kg (of body weight). Before treatment and one hour after treatment with test article, monitoring was performed at 2 minute intervals within the first 10 minutes and then at 10 minute intervals. The rats were monitored individually—RR, HR, BRT, and AN, AT, CR, PRT, PRToe, RRfe, ST.

The time period required to induce fentanyl overdose was measured from administration to suppression of all monitored reflexes and a significant reduction in respiratory (risk of respiratory arrest) and cardiac frequency. At that time, the test article was administered intranasally and began recording the time required for complete recovery of all reflexes, and consequent increase in heart rate and respiratory rate.

Total recovery time was defined as time needed to return all overdose parameters (RR, HR, AN—Alertness, AT—Astasia, (Astasia-abasia is defined as the inability to stand and to walk, despite sparing of motor function underlying the required balance and gestures) CR—Corneal reflex, PRT—Pinch reflex tail, PRToe—Pinch reflex toe, Rref—Righting reflex, ST—Sternal recumbency) to normal physiological values and at least of 67% of resting respiratory rate.

FIGS. 1A-1D is a collection of four graphs showing the results of measurements of the effects of pharmacological substances on vital signs and reflexes. FIG. 1A. shows the design of the experiment and the vitals and reflexes monitored: RR—respiratory rate, HR—heart rate, BT—body temperature, AN—alertness, AT—astasia, CR—corneal reflex, PRT—pinch reflex tail, PRToe—pinch reflex toe, Rref—righting reflex, ST—sternal recumbency.

FIG. 1B. depicts the mean time to all reflexes restored at 0.31 and 0.62 μmol/kg doses, while FIG. 1C. shows the mean time to all reflexes restored at 1.24 μmol/kg doses. FIG. 1D illustrates RR AUC (RR×min) for each treated group. Resting RR AUC was calculated by multiplying average resting RR by 60 min.

After achieving an overdose in each individual animal, one of the following naloxone (NX), NX90, NB33 and NX90+NB33 was administered on equimolar basis (FIG. 1A). As previously published, we qualified an overdose reversal as restoration of all monitored reflexes and respiratory rate (RR) to at least 67% of the animal's resting RR¹⁴. The mean time to overdose reversal defined in Methods for each treatment group is shown on FIG. 1B. We found that for all test articles the overdose reversal was dose dependent, with the mean reversal time decreasing as follows: NB33>NX>NX90>NX90+NB33 for low 0.31 umol/kg and high 0.62 umol/kg doses. Accordingly, for low dose the mean reversal times were: 1548±173, 684±159, 372±90 and 114±68 seconds. For the high dose, the mean reversal times were: 1272±278, 288±130, 102±58 and 48±7 seconds. Surprisingly, the coadministration of NX90 with much less effective NB33 was not only 5-fold more effective than naloxone alone but also 2-fold more effective than NX90 alone. In fact, rats treated with a combination of NX90 and NB33 demonstrated complete recovery of all measured reflexes (e.g. “jumping”) within 30 seconds of intranasal administration. On one hand, this results strongly suggest that κ-agonism plays a role in OD reversal and may further complement our current understanding of robustness of opioid reversal through antidote's affinity for and kinetics of association and dissociation with μ-receptor¹⁰. On the other hand, a ratio of net μ-antagonism and κ-agonism may be important as NB33 alone was not an effective OD reversal agent at any dose.

Since this data was obtained from equimolar doses, one could suggest that we are observing an additive effect of μ-antagonism when both NX90 and NB33 were co-administered. To challenge this explanation, we compared even higher 1.24 umol/kg doses of naloxone and NX90 against the ½ equimolar doses of NX90+NB33 (FIG. 1D). Noteworthy, the 1.24 umol/kg dose of naloxone allometrically corresponds to 5.3 mg intranasal dose per adult human that is significantly higher that the recommended dose of 2 mg³³. At this higher dose, full recovery in the naloxone group was observed in 96±24 seconds that is close to the fastest recovery possible under the current standard of care. Interestingly, for the equimolar dose of NX90 the recovery time was only slightly faster—84±24 seconds, perhaps indicating the limitations of the model for mostly μ-antagonists. However, the 1₂ equimolar dose of the NX90+NB33 combination produced much more robust recovery in 48±7 seconds (FIG. 1D). Therefore, the observed synergistic effect cannot be explained simply by additive μ-antagonism. This led us to believe that the observed synergy of NX90+NB33 must have come from the activation of κ-opioid receptors. However, a much weaker μ-antagonist with pronounced x-agonistic properties NB33 failed to improve recovery time. This suggest that the synergistic activity may have come from NB33 potentially outcompeting much stronger μ-antagonist NX90 at κ-opioid receptors and increasing concentration of the latter at μ-opioid receptors.

Following our methodology¹⁴, we also assessed the total severity of respiratory depression RR AUC (RR×min) as a cumulative measure of respiratory activity to quantify a risk burden for hypoxic injury to the brain²⁷ that could lead to severe cognitive aberrations and abandoning safe injection practices in people who use opioids²⁸. We found it to be significantly lower for NX90 and NX90+NB33 treated animals compared to the naloxone or NB33 alone groups (FIG. 1B).

TABLE 1
Naloxone testing and recovery time evaluation

| | | | | | | | Average | Dose/rat | ml of | Total | |
|---|---|---|---|---|---|---|---|---|---|---|---|
| | Tested dose | | | Body | Fentanyl | Overdose | overdose | NX90:NB33 | diluted | recovery | Average total |
| | μmol/kg | Rat | | weight | Dose/rat | induction | time | (0.4 mg:0.4 mg) | NX90:NB33 | time | recovery time |
| Group | (mpk) | no | Sex | (kg) | (mg) | (seconds) | (seconds) | (mg) | IN | (seconds) | (seconds) |
| NX | 0.16 (0.1) | 1 | M | 0.271 | 0.0352 | 180 | 168 | 0.0271 | 0.0678 | 420 | 684 ± 356 |
| 1 | | 2 | F | 0.235 | 0.0306 | 60 | (2 min and | 0.0235 | 0.0588 | 540 | |
| | | 3 | F | 0.243 | 0.0316 | 240 | 48 sec) | 0.0243 | 0.0608 | 1200 | |
| | | 4 | M | 0.243 | 0.0316 | 180 | | 0.0243 | 0.0608 | 900 | |
| | | 5 | F | 0.220 | 0.0286 | 180 | | 0.0220 | 0.0550 | 360 | |
| NX | 0.32 (0.2) | 1 | M | 0.358 | 0.0465 | 120 | 132 | 0.0716 | 0.1790 | 300 | 288 ± 130 |
| 2 | | 2 | M | 0.327 | 0.0425 | 60 | (2 min and | 0.0654 | 0.1635 | 480 | |
| | | 3 | F | 0.329 | 0.0428 | 240 | 12 sec) | 0.0658 | 0.1645 | 120 | |
| | | 4 | M | 0.415 | 0.0540 | 120 | | 0.0830 | 0.2075 | 300 | |
| | | 5 | F | 0.304 | 0.0395 | 120 | | 0.0608 | 0.1520 | 240 | |

TABLE 2
NX90 testing and recovery time evaluation

| | | | | | | | Average | Dose/rat | ml of | Total | |
|---|---|---|---|---|---|---|---|---|---|---|---|
| | Tested dose | | | Body | Fentanyl | Overdose | overdose | NX90:NB33 | diluted | recovery | Average total |
| | μmol/kg | Rat | | weight | Dose/rat | induction | time | (0.4 mg:0.4 mg) | NX90:NB33 | time | recovery time |
| Group | (mpk) | no. | Sex | (kg) | (mg) | (seconds) | (seconds) | (mg) | IN | (seconds) | (seconds) |
| NX90 | 0.16 (0.13) | 1 | F | 0.261 | 0.0339 | 240 | 288 | 0.0339 | 0.0424 | 360 | 372 ± 201 |
| 1 | | 2 | M | 0.270 | 0.0351 | 360 | (4 min and | 0.0351 | 0.0439 | 240 | |
| | | 3 | M | 0.274 | 0.0356 | 360 | 48 sec) | 0.0356 | 0.0445 | 720 | |
| | | 4 | F | 0.263 | 0.03.42 | 360 | | 0.0342 | 0.0427 | 240 | |
| | | 5 | M | 0.278 | 0.0361 | 120 | | 0.0361 | 0.0452 | 300 | |
| NX90 | 0.32 (0.26) | 1 | M | 0.398 | 0.0517 | 120 | 312 | 0.1035 | 0.1294 | 60 | 102 ± 58 |
| 2 | | 2 | M | 0.374 | 0.0486 | 480 | (5 min and | 0.0972 | 0.1216 | 30 | |
| | | 3 | F | 0.344 | 0.0447 | 360 | 12 sec) | 0.0894 | 0.1118 | 120 | |
| | | 4 | F | 0.384 | 0.0499 | 240 | | 0.0998 | 0.1248 | 180 | |
| | | 5 | M | 0.385 | 0.0501 | 360 | | 0.1001 | 0.1251 | 120 | |
| NX90 | 0.64 (0.52) | 1 | M | 0.249 | 0.0324 | 240 | 228 | 0.1295 | 0.1619 | 180 | 84 ± 54 |
| 3 | | 2 | F | 0.221 | 0.0287 | 360 | (3 min and | 0.1149 | 0.1437 | 60 | |
| | | 3 | M | 0.232 | 0.0302 | 180 | 48 sec) | 0.1206 | 0.1508 | 60 | |
| | | 4 | M | 0.235 | 0.0306 | 240 | | 0.1222 | 0.1528 | 60 | |
| | | 5 | F | 0.201 | 0.0261 | 120 | | 0.1045 | 0.1307 | 60 | |

TABLE 3
NX90 + NB33 testing and recovery time evaluation

| | | | | | | | Average | Dose/rat | ml of | Total | |
|---|---|---|---|---|---|---|---|---|---|---|---|
| | Tested dose | | | Body | Fentanyl | Overdose | overdose | NX90:NB33 | diluted | recovery | Average |
| | μmol/kg | Rat | | weight | Dose/rat | induction | time | (0.4 mg:0.4 mg) | NX90:NB33 | time | recovery time |
| Group | (mpk | no. | Sex | (kg) | (mg) | (seconds) | (seconds) | (mg) | IN | (seconds) | (seconds) |
| NX90 + | 0.16 + 0.16 | 1 | F | 0.319 | 0.0415 | 120 | 240 | 0.0829 | 0.1037 | 180 | 114 ± 68 |
| NB33 | | 2 | M | 0.415 | 0.0540 | 240 | (4 min and | 0.1079 | 0.1349 | 180 | |
| 1 | | 3 | M | 0.385 | 0.0501 | 240 | 0 sec) | 0.1001 | 0.1251 | 60 | |
| | | 4 | M | 0.329 | 0.0428 | 360 | | 0.0855 | 0.1069 | 30 | |
| | | 5 | F | 0.320 | 0.0416 | 240 | | 0.0832 | 0.1040 | 120 | |
| NX90 + | 0.32 + 0.32 | 1 | M | 0.276 | 0.0359 | 360 | 264 | 0.1435 | 0.1794 | 30 | 48 ± 16 |
| NB33 | | 2 | M | 0.259 | 0.0337 | 240 | (4 min and | 0.1347 | 0.1684 | 60 | |
| 2 | | 3 | F | 0.241 | 0.0313 | 240 | 24 sec) | 0.1253 | 0.1567 | 60 | |
| | | 4 | F | 0.222 | 0.0289 | 240 | | 0.1154 | 0.1443 | 30 | |
| | | 5 | F | 0.225 | 0.0293 | 240 | | 0.1170 | 0.1463 | 60 | |

TABLE 4
Data obtained by group and sex, regarding the average recovery time and the
percentage of MPA (maximum possible analgesia) for NX + NB combinations

| | | | | Sex and | Average | | | | | | **MPA % | |
|---|---|---|---|---|---|---|---|---|---|---|---|---|
| | Drug | Group | Dose | number | recovery time | | AT − 20 TFT* | | PT + 40 TFT* | | average | **MPA % |
| | tested | no. | mg/kg | of rats | seconds | | seconds | | seconds | | by sex | average |
| 1 | NB | 1. NB | 0.1 | 5 M | 5760 | 5400 | 2.57 | 2.62 | >40 | >40 | 100 | 100 |
| | | | | 5 F | 5040 | | 2.67 | | >40 | | 100 | |
| 2 | NX:NB | 2. NX:NB | 0.1:0.1 | 3 M | 180 | 210 | 3.00 | 2.80 | 12.09 | 9.57 | 24.57 | 18.18 |
| | 1:1 | | | 2 F | 240 | | 2.61 | | 7.04 | | 11.86 | |
| 3 | NX:NB | 3. NX:NB | 0.05:0.05 | 3 M | 250 | 230 | 3.21 | 3.18 | 12.01 | 11.12 | 23.91 | 21.57 |
| | 1:1 | | | 2 F | 210 | | 3.15 | | 10.24 | | 19.24 | |
| 4 | NX:NB | 4. NX:NB | 0.1:0.025 | 2 M | 157.5 | 213.75 | 3.28 | 3.05 | 5.49 | 5.37 | 6.03 | 6.29 |
| | 1:0.25 | | | 3 F | 270 | | 2.83 | | 5.26 | | 6.54 | |
| 5 | NX:NB | 5. NX:NB | 0.1:0.4 | 2 M | 180 | 125 | 2.44 | 2.45 | 22.79 | 23.83 | 54.17 | 56.94 |
| | 1:4 | | | 3 F | 70 | | 2.46 | | 24.88 | | 59.71 | |

Predetermined cutoff time of 40 seconds was used
*AT − 20 TFT = ante therapeutic average TFT time (seconds), 20 minutes before fentanyl administration
*PT + 40 TFT = post therapeutic average TFT time (seconds), 40 minutes after fentanyl administration
**Percentage of maximum possible analgesia (% MPA) using the formula: % MPA = [(Test − Baseline)/(Cutoff − Baseline)] × 100%

TABLE 5
Functional profile of NX90, NX33 and parent opioids
in CHO transfected cells.

| | μ-receptor | k-receptor | k-receptor | δ-receptor |
|---|---|---|---|---|
| | inhibition | inhibition | activation | inhibition |
| | (IC50 or %) | (IC50 or %) | (IC50 or %) | (IC50 or %) |
| NX90 | 137 ± 12 nM | 2,020 ± 640 nM | 50 ± 15 nM | >10 μM |
| Naloxone | 9.8 ± 2.0 nM | 48 ± 13 nM | — | 33.7 ± 7.8 |
| NB33 | 82.8 ± 4.1% @1uM | — | 22.3 ± 9.1% @1uM | >10 μM |
| Nalbuphine | 82.6 ± 3.4% @1uM | — | 69.1 ± 7.4% @1uM | >10 μM |

These assertions are consistent with the apparent congruency of the PK profiles of released parent opioids (Table 6). Furthermore, since both NX90 and NB33 were mostly intact in brain homogenate (85±13% and65±6%; data not shown) within the time frame of the study, the coadministration of both agents should have resulted in substantially overlapping levels in the brain and in overall additional net κ-agonism exposure.

TABLE 6
Mean (±SD) concentration of parent opioids in plasma after IV administration of
NX90 or NX33.

| | Dose | t_{1/2} | C₀ | AUC_{Inf} | MRT | Vss | CL |
|---|---|---|---|---|---|---|---|
| Group | μmol/kg | (h) | (ng/mL) | (h*ng/mL) | (h) | (L/kg) | (mL/min/kg) |
| NX90 | 3.1 | 0.37 ± 0.04 | 348 ± 48 | 84 ± 5 | 0.35 ± 0.04 | 6 ± 1 | 260 ± 16 |
| NB33 | 3.1 | 0.23 ± 0.05 | 1136 ± 72 | 346 ± 35 | 0.30 ± 0.06 | 3 ± 1 | 179 ± 17 |

### Example 2—Safety Assessment of NX90+NB33 Combinations Vs. Naloxone

Naloxone administration is associated with catecholamine release that is thought to be involved in pulmonary edema¹⁰ and marked cardiovascular stimulation¹¹ the most prevalent side effects in overdose patients. These may lead to serious adverse events reported for doses as low as 2 μg/kg and raise a question what would be a safe dose in patients susceptible to naloxone induced withdrawal. Since naloxone-induced withdrawal could be characterized by activation of catecholaminergic neurons in the heart, we evaluated changes in the heart rate (HR) as a proxy for catecholamine surge.

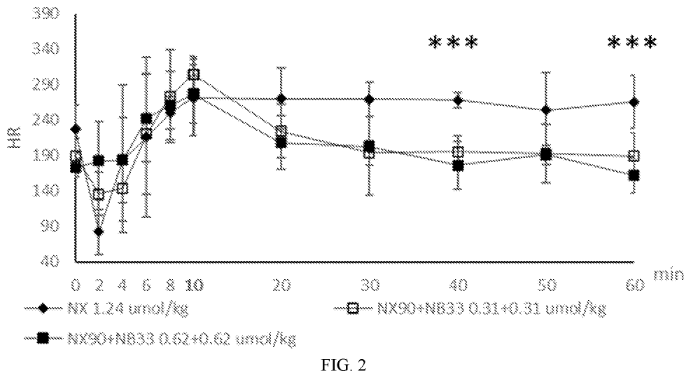

FIG. 2. depicts the mean heart rate (HR) for the naloxone (1.24 μmol/kg), NX90 (1.24 μmol/kg) or NB33+NX90 (0.31+0.31 μmol/kg and 0.62+0.62 μmol/kg) treated groups (n=5) in fentanyl driven OD model. We evaluated effects of fentanyl on HR as a common contributor to heart rate among all treated groups (FIG. 2). In our hands, fentanyl did not have any dose dependent effect on the heart rate over span of two hours that is consistent with previously published reports (Baechtold et al., 2001). At the highest dose of fentanyl (0.13 mpk) that was used in our OD study there was high animal-to-animal variability in HR with non-significant overall elevation. As expected, HR in all naloxone treated groups was elevated without clear dose-dependance and stayed above the resting HR at all timepoints by about 74 bpm on average (FIG. 2).

Either dose of NX90 had a bell-shaped response with HR returning to the resting values at 50-60 min time points. On the other hand, NB33 effect on HR was similar to that of naloxone. Finally, we compared the highest doses of naloxone with NX90+NB33 (0.31+0.31 μmol/kg and 0.62+0.62 μmol/kg) treated animals. Surprisingly, heart rate (HR) elevation in animals treated with combinations (e.g., NX90+NB33) at all doses, proved to be transient and returned to resting HR rates within 30 min unlike animals treated with Naloxone.

### Example 3—Dose Ratio Comparison

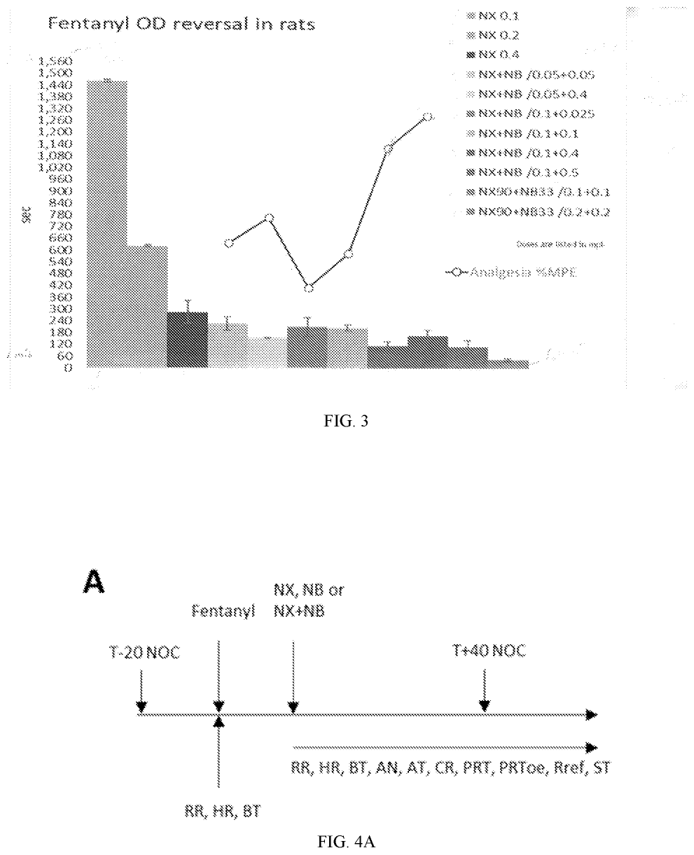

FIG. 3 is a bar graph depicting dose rate comparisons of types and quantities of the test substances on OD reversal in subject rats. Experiments were conducted to compare efficacy of doses of naloxone administered to rats given both separately and in combination with various ratios of doses of nalbuphine and naloxone, or NB33 and NX90. The bar graph in FIG. 3 displays the effect of various doses of naloxone alone or in combination with nalbuphine on fentanyl overdose reversal in rats. Reversal time is measured in seconds.

As mentioned above, NB33 is a 3-hexadienoate derivative of nalbuphine that converts to the parent drug in a biological matrix, while NX90 is a 3-hexadienoate derivative of Naloxone that converts to the parent drug in a biological matrix. Surprisingly, lower doses of NB33 (0.1 mg/kg, 0.2 mg/kg) in combination with 0.1 mg/kg of NX90 reversed fentanyl overdose in rats in 1-2 minutes.

Thus, it is seen that the objects of the invention are efficiently obtained, although changes and modifications to the invention should be readily apparent to those having ordinary skill in the art, which changes would not depart from the spirit and scope of the invention as claimed.

### Example 4—Evaluation of NX, NB and their Combinations Efficacy in Fentanyl-Induced Overdose Model

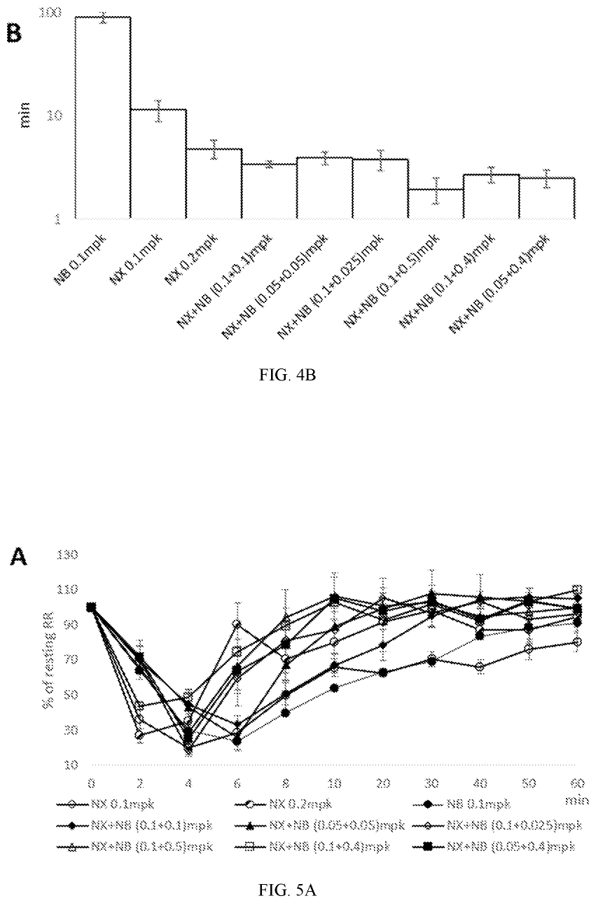

Using the optimal fentanyl dose (see Materials and Methods), sedation/analgesia were installed in rats that were monitored individually for respiratory rate (RR), heart rate (HR), and body temperature (BT), as well as eight basic reflexes including nociception (FIG. 4A).

After achieving an overdose in each individual animal, one of the following NX, NB or NX+NB combination was administered. We have qualified an overdose reversal as full restoration of all monitored reflexes and respiratory rate (RR) to at least 67% of the animal's resting RR.¹⁴ The mean time to overdose reversal defined in Methods for each treatment group is shown in FIG. 4B. We found that NB was less effective in reversing a fentanyl induced overdose when compared to NX at both tested doses (0.1 and 0.2 mg/kg) with the mean overdose reversal times were 90±9.9, 11.4±2.7 and 4.8±1.0 minutes correspondingly.

However, we observed a statistically significant synergistic effects on overdose reversal when a combination of NX+NB was administered. Accordingly, the mean reversal times for the combinations ranged from 3.9±0.6 min to 1.95±0.5 min with a combination of NX+NB (0.1+0.5 mg/kg) producing the fastest overdose reversal of all measured reflexes (FIG. 4B). It is quite surprising, as one would expect that co-administration of NB would result in lower alertness—a common side effect²² that could be detrimental to effective overdose reversal. In fact, the alertness in the NB only treated group was the last or next to last reflex to recover. However, alertness in all combination groups even at the highest dose of NB (0.5 mg/kg) was the same or better as in NX only treated groups.

Interestingly, supplementing NX with even 0.025 mg/kg of NB produced a significantly faster reversal with 3.75±0.8 min than any tested dose of NX alone. Furthermore, a sub-therapeutic dose (0.05 mg/kg) of NX when supplemented by 0.05 mg/kg of NB, was more effective than any tested dose of NX alone with the reversal time of 3.9±0.6 min. Therefore, the observed synergistic effect on overdose reversal cannot be explained simply by additive μ-antagonism from co-administration of NB and is likely that κ-opioid receptors are playing a role in it.

### Example 5—Evaluation of Respiratory and Cardiovascular Liability of NX, NB and their Combination in Fentanyl-Induced Overdose Model

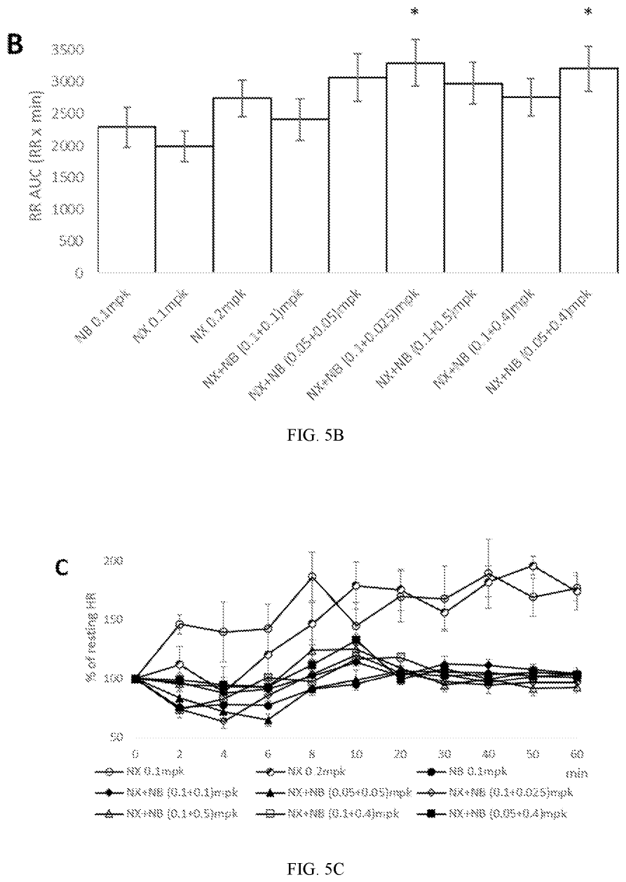

The main risk of death from a fentanyl overdose comes from respiratory depression. Analysis of respiratory rates calculated as percent of resting rates (FIG. 5A) showed that under most of interventions, respiratory rates normalized within 6-8 minutes.¹⁴ However, 0.1 mg/kg of NB or NX alone were the least effective interventions when respiratory rate reversal was achieved only within 10-20 min.

However, fatal overdoses constitute only 2.64% of all overdose events.^{25,26} Therefore the risk of hypoxic injury to the brain²⁷ as a result of respiratory depression that could lead to long term disability²⁸ is also an important indicator of effectiveness of overdose reversal. With respiratory depression produced by usual therapeutic doses of NB is equivalent to that of morphine,²⁹ one could expect an increased risk for hypoxic injury from a combination of NX+NB. To quantify a risk burden for the hypoxic injury, we examined RR AUC (RR×min) as cumulative measure of respiratory activity (FIG. 5B). To our surprise, we found that supplementation of NX with NB led to actual reduction in the total severity of respiratory depression. Moreover, two combinations NX+NB (0.1+0.025 mg/kg and 0.05+0.4 mg/kg) worked significantly better that NX alone, increasing the respective RR AUC by about 1.6-fold. With no clear dose dependence, we concluded that co-administration of NX with NB does not increase the risk of hypoxic injury, but provides a mild improvement in the respiratory rate.

In addition, as naloxone-induced withdrawal could be characterized by the activation of catecholaminergic neurons in the heart, we evaluated changes in the heart rate (HR) as a proxy for catecholamine surge.^{11,30} As expected, we found that HR in both naloxone treated groups was elevated and stayed above the resting HR at all timepoints (FIG. 5C). However, supplementation with NB completely mitigated this negative effect. When we looked at HR AUC (HR×min), a cumulative measure of heart rate, we found that compared to naloxone alone, all combinations resulted in statistically lower times for restoring HR (FIG. 5D). This data suggests that κ-agonism could potentially mitigate the severity of cardiorespiratory liabilities of naloxone.⁹

### Example 6—Evaluation of Net Analgesia of Nx, Nb and their Combination in Fentanyl-Induced Overdose Model

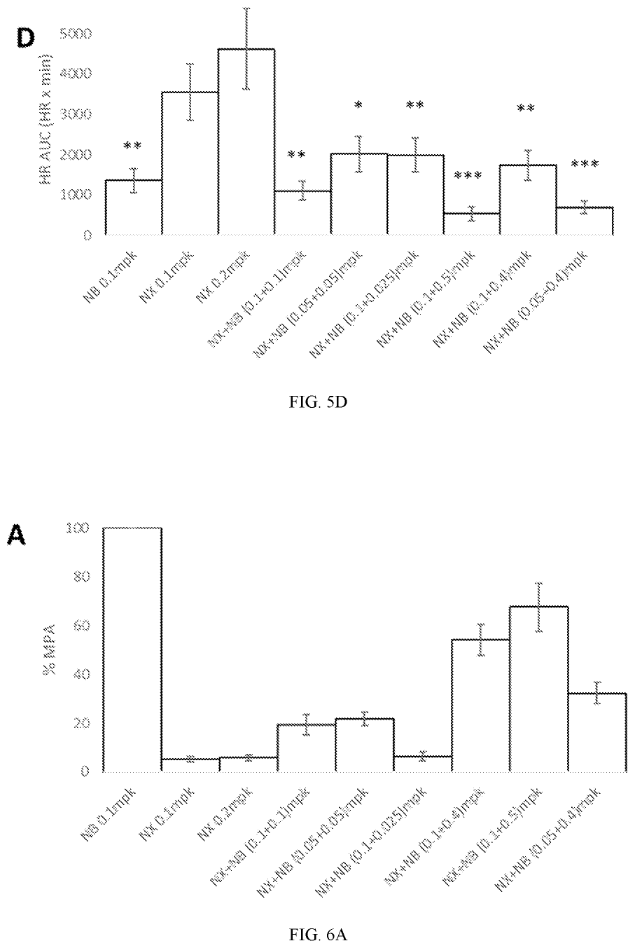

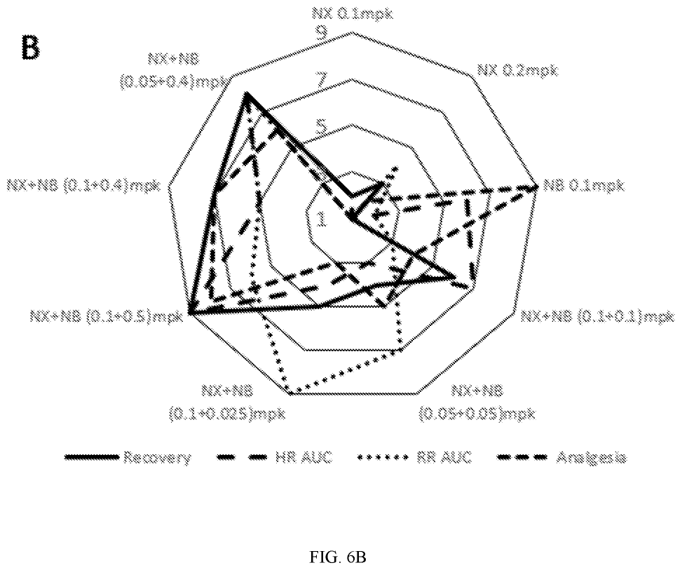

We evaluated the potential of various NX+NB combinations to address hyperalgesia and lower pain tolerance associated with opioid withdrawal. We measured latency of tail flick to thermal stimuli^{31,32} in rats before fentanyl administration (T−20 min) as a baseline and 40 min after intervention (FIG. 6A). FIG. 3 shows data for each treated group as percent of maximum possible analgesia (% MPA). We found residual net analgesia (˜5%) in NX treated groups with MPA of 100% (exceeding 40 sec predetermined cutoff) in NB treated groups. For all combinations, the increase in % MPA was a NB dose dependent rather than a ratio dependent (FIG. 6A). Also, for a combination of drugs there was a threshold dose of NB (0.05 mg/kg) to produce a significant improvement in analgesia, as a combination with lower dose of NB (0.025 mg/kg) had % MPA similar to that of NX only treated animals.

Finally, we rank-ordered all interventions for overdose reversal potential, cumulative risks for cardiorespiratory liabilities, and analgesia with the lowest rank of 1 in the center of the graph (FIG. 6B). All NX+NB combinations outranked all doses of NX only for the evaluated endpoints. Even combinations of sub-therapeutic dose of NX (0.05 mg/kg) together with NB (0.05 or 0.4 mg/kg) outperformed the highest dose (0.2 mg/kg) of NX only. This finding indicates that with a combination therapy not only faster overdose reversal could be achieved but also the higher doses of NX can be avoided to prevent the associated side effects.

### REFERENCES

1. France C P, Ahern G P, Averick S, et al. Countermeasures for Preventing and Treating Opioid Overdose. Clin Pharmacol Ther. 2021; 109(3):578-590. doi:10.1002/cpt.2098
2. Bisaga A. What should clinicians do as fentanyl replaces heroin? Addiction. 2019; 114(5):782-783. doi:10.1111/add.14522
3. Bennett A S, Freeman R, Des Jarlais D C, Aronson I D. Reasons People Who Use Opioids Do Not Accept or Carry No-Cost Naloxone: Qualitative Interview Study. JMIR Form Res. 2020; 4(12):e22411. doi:10.2196/22411
4. Lai J T, Goldfine C E, Chapman B P, et al. Nobody Wants to Be Narcan'd: A Pilot Qualitative Analysis of Drug Users' Perspectives on Naloxone. West J Emerg Med. 2021; 22(2):339-345. doi:10.5811/westjem.2020.10.48768
5. Shah M, Huecker M R. Opioid Withdrawal. In; 2022.
6. Neale J, Strang J. Naloxone—does over-antagonism matter? Evidence of iatrogenic harm after emergency treatment of heroin/opioid overdose. Addiction. 2015; 110(10):1644-1652. doi:10.1111/add.13027
7. Weiner S G, Baker O, Bernson D, Schuur J D. One-Year Mortality of Patients After Emergency Department Treatment for Nonfatal Opioid Overdose. Ann Emerg Med. 2020; 75(1):13-17. doi:10.1016/j.annemergmed.2019.04.020
8. Uddin O, Jenne C, Fox M E, Arakawa K, Keller A, Cramer N. Divergent profiles of fentanyl withdrawal and associated pain in mice and rats. Pharmacol Biochem Behav. 2021; 200:173077. doi:10.1016/j.pbb.2020.173077
9. van Dorp E L A, Yassen A, Dahan A. Naloxone treatment in opioid addiction: the risks and benefits. Expert Opin Drug Saf 2007; 6(2):125-132. doi:10.1517/14740338.6.2.125
10. Rzasa Lynn R, Galinkin J L. Naloxone dosage for opioid reversal: current evidence and clinical implications. Ther Adv drug Saf. 2018; 9(1):63-88. doi:10.1177/2042098617744161
11. Kienbaum P, Scherbaum N, Thürauf N, Michel M C, Gastpar M, Peters J. Acute detoxification of opioid-addicted patients with naloxone during propofol or methohexital anesthesia: a comparison of withdrawal symptoms, neuroendocrine, metabolic, and cardiovascular patterns. Crit Care Med. 2000; 28(4):969-976. doi:10.1097/00003246-200004000-00010
12. Zuurmond W W, Meert T F, Noorduin H. Partial versus full agonists for opioid-mediated analgesia—focus on fentanyl and buprenorphine. Acta Anaesthesiol Belg. 2002; 53(3):193-201.
13. Yassen A, Olofsen E, van Dorp E, et al. Mechanism-Based Pharmacokinetic-Pharmacodynamic Modelling of the Reversal of Buprenorphine-Induced Respiratory Depression by Naloxone. Clin Pharmacokinet. 2007; 46(11):965-980. doi:10.2165/00003088-200746110-00004
14. Voronkov M, Nikonov G, Ataiants J, et al. Modifying naloxone to reverse fentanyl-induced overdose. Int J Pharm. Published online November 2021:121326. doi:10.1016/j.ijpharm.2021.121326
15. Voronkov M, Ocheret D, Bondarenko S, Yu Y, Koren S. Administration of Nalbuphine to Heroin Addicts. Feasibility and Short-Term Effects. Heroin Addict Relat Clin Probl. 2008; 10.
16. Wang Y hua, Sun J feng, Tao Y min, Chi Z giang, Liu J gen. The role of κ-opioid receptor activation in mediating antinociception and addiction. Acta Pharmacol Sin. 2010; 31(9):1065-1070. doi:10.1038/aps.2010.138
17. Spanagel R, Almeida O F, Bartl C, Shippenberg T S. Endogenous kappa-opioid systems in opiate withdrawal: role in aversion and accompanying changes in mesolimbic dopamine release. Psychopharmacology (Berl). 1994; 115(1-2):121-127. doi:10.1007/BF02244761
18. Moss R B, Pryor M M, Baillie R, et al. Higher naloxone dosing in a quantitative systems pharmacology model that predicts naloxone-fentanyl competition at the opioid mu receptor level. PLoS One. 2020; 15(6):e0234683. doi:10.1371/journal.pone.0234683
19. Voronkov M, Nikonov G, Naumov R, Abernethy J, Isakulyan L. NB-33, a bioreversible opioid derivative of Nalbuphine, shows enhanced pharmacodynamics. Eur J Pharm Sci Off J Eur Fed Pharm Sci. 2021; 159:105734. doi:10.1016/j.ejps.2021.105734
20. Raynor K, Kong H, Chen Y, et al. Pharmacological characterization of the cloned kappa-, delta-, and mu-opioid receptors. Mol Pharmacol. 1994; 45(2):330-334.
21. Peng X, Knapp B I, Bidlack J M, Neumeyer J L. Pharmacological properties of bivalent ligands containing butorphan linked to nalbuphine, naltrexone, and naloxone at mu, delta, and kappa opioid receptors. J Med Chem. 2007; 50(9):2254-2258. doi:10.1021/jm061327z
22. Schmidt W K, Tam S W, Shotzberger G S, Smith D H J, Clark R, Vernier V G. Nalbuphine. Drug Alcohol Depend. 1985; 14(3-4):339-362. doi:10.1016/0376-8716(85)90066-3
23. Gear R W, Gordon N C, Miaskowski C, Paul S M, Heller P H, Levine J D. Dose ratio is important in maximizing naloxone enhancement of nalbuphine analgesia in humans. Neurosci Lett. 2003; 351(1):5-8. doi:10.1016/s0304-3940(03)00939-x
24. Schmidt B L, Gear R W, Levine J D. Response of neuropathic trigeminal pain to the combination of low-dose nalbuphine plus naloxone in humans. Neurosci Lett. 2003; 343(2):144-146. doi:10.1016/s0304-3940(03)00356-2
25. Stoové MA, Dietze P M, Jolley D. Overdose deaths following previous non-fatal heroin overdose: Record linkage of ambulance attendance and death registry data. Drug Alcohol Rev. 2009; 28(4):347-352. doi:10.1111/j.1465-3362.2009.00057.x
26. Darke S, Mattick R P, Degenhardt L. The ratio of non-fatal to fatal heroin overdose. Addiction. 2003; 98(8):1169-1171. doi:10.1046/j.1360-0443.2003.00474.x
27. Voronkov M, Cocchiaro B, Stock J B. Does a hypoxic injury from a non-fatal overdose lead to Alzheimer Disease? Neurochem Int. 2021; 143:104936. doi:10.1016/j.neuint.2020.104936
28. Voronkov M, Ataiants J, Cocchiaro B, Stock J B, Lankenau S E. A vicious cycle of neuropathological, cognitive and behavioural sequelae of repeated opioid overdose. Int J Drug Policy. 2021; 97:103362. doi:10.1016/j.drugpo.2021.103362
29. Miller R R. Evaluation of nalbuphine hydrochloride. Am J Hosp Pharm. 1980; 37(7):942-949.
30. Rabadán J V, Milanés M V, Laorden M L. Changes in right atrial catecholamine content in naïve rats and after naloxone-induced withdrawal. Br J Anaesth. 1998; 80(3):354-359. doi:10.1093/bja/80.3.354
31. Wang J J, Ho S T, Hu O Y, Chu K M. An innovative cold tail-flick test: the cold ethanol tail-flick test. Anesth Analg. 1995; 80(1):102-107. doi:10.1097/00000539-199501000-00018
32. Chu K S, Wang J J, Yoa-Pu Hu O, Ho S T, Chen Y W. The antinociceptive effect of nalbuphine and its long-acting esters in rats. Anesth Analg. 2003; 97(3):806-809. doi:10.1213/01.ANE.0000078589.90903.EF
33. Lavonas E J, Drennan I R, Gabrielli A, Heffner A C, Hoyte C O, Orkin A M, Sawyer K N, Donnino M W. Part 10: Special Circumstances of Resuscitation: 2015 American Heart Association Guidelines Update for Cardiopulmonary Resuscitation and Emergency Cardiovascular Care. Circulation. 2015 Nov. 3; 132(18 Suppl 2):5501-18. doi: 10.1161/CIR.0000000000000264. Erratum in: Circulation. 2016 Aug. 30; 134(9):e122. PMID: 26472998.

We claim:

1. A composition comprising:
a therapeutically effective dose of an opioid receptor mu antagonist; and,
a therapeutically effective dose of an opioid receptor kappa agonist.

2. The composition of claim 1, wherein the opioid receptor mu antagonist is naloxone or a derivative thereof and the opioid receptor kappa agonist is nalbuphine or a derivative thereof.

3. The composition of claim 2, wherein the derivative of naloxone is a 3-hexadienoate derivative of naloxone (NX90) and the derivative of nalbuphine is a 3-hexadienoate derivative of nalbuphine (NB33).

4. The composition of claim 1, wherein the opioid receptor mu antagonist is NX90, and the opioid receptor kappa agonist is NB33; or
wherein the opioid receptor mu antagonist is naloxone, and the opioid receptor kappa agonist is NB33; or
wherein the opioid receptor mu antagonist is NX90, and the opioid receptor kappa agonist is nalbuphine; or
wherein the opioid receptor mu antagonist is naloxone, and the opioid receptor kappa agonist is nalbuphine.

5. The composition of claim 1, wherein the therapeutically effective dose of the opioid receptor mu antagonist is about 0.05 mg/kg to about 0.2 mg/kg body weight of the subject; and the therapeutically effective dose of the opioid receptor kappa agonist is about 0.025 mg/kg to about 2.5 mg/kg body weight of the subject, is about 0.025 mg/kg to about 2.0 mg/kg body weight of the subject, is about 0.025 mg/kg to about 1.5 mg/kg body weight of the subject, is about 0.025 mg/kg to about 1.0 mg/kg body weight of the subject, or is about 0.025 mg/kg to about 0.5 mg/kg body weight of the subject.

6. The composition of claim 5, wherein the therapeutically effective dose of the opioid receptor mu antagonist is about 0.1 mg/kg to about 0.2 mg/kg body weight of the subject; and the therapeutically effective dose of the opioid receptor kappa agonist is about 0.05 mg/kg to about 0.4 mg/kg body weight of the subject, or is about 0.025 mg/kg to about 0.1 mg/kg body weight of the subject.

7. The composition of claim 1, wherein the therapeutically effective dose of the opioid receptor mu antagonist is the same as the therapeutically effective dose of the opioid receptor kappa agonist.

8. The composition of claim 7, wherein the therapeutically effective dose of the opioid receptor mu antagonist and the opioid receptor kappa agonist is about 0.05 mg/kg to about 2.5 mg/kg.

9. The composition of claim 1, wherein the therapeutically effective dose of the opioid receptor mu antagonist is at a ratio of about 4:1 to about 1:8 relative to the therapeutically effective amount of the opioid receptor kappa agonist.

10. The composition of claim 9, wherein the therapeutically effective dose of the opioid receptor mu antagonist is at a ratio of about 4:1, about 1:4, about 1:5, or about 1:8 relative to the therapeutically effective amount of the opioid receptor kappa agonist.

11. The composition of claim 10, wherein the wherein the therapeutically effective dose of the opioid receptor mu antagonist is about 0.05 mg/kg to about 0.1 mg/kg.

12. A method of blocking an opioid from binding to an opioid receptor in a subject, the method comprising administering to the subject a composition comprising a therapeutically effective dose of an opioid receptor mu antagonist and a therapeutically effective dose of an opioid receptor kappa agonist.

13. The method of claim 12, wherein the opioid receptor mu antagonist is naloxone or a derivative thereof, and the opioid receptor kappa agonist is nalbuphine or a derivative thereof.

14. The method of claim 13, wherein the derivative of naloxone is a 3-hexadienoate derivative of naloxone (NX90), and the derivative of nalbuphine is a 3-hexadienoate derivative of nalbuphine (NB33).

15. The method of claim 12, wherein the opioid receptor mu antagonist is NX90, and the opioid receptor kappa agonist is NB33; or wherein the opioid receptor mu antagonist is naloxone, and the opioid receptor kappa agonist is NB33; or wherein the opioid receptor mu antagonist is NX90, and the opioid receptor kappa agonist is nalbuphine; or wherein the opioid receptor mu antagonist is naloxone, and the opioid receptor kappa agonist is nalbuphine.

16. The method of claim 12, wherein the therapeutically effective dose of the opioid receptor mu antagonist is about 0.05 mg/kg to about 0.2 mg/kg body weight of the subject; and the therapeutically effective dose of the opioid receptor kappa agonist is about 0.025 mg/kg to about 2.5 mg/kg body weight of the subject, is about 0.025 mg/kg to about 2.0 mg/kg body weight of the subject, is about 0.025 mg/kg to about 1.5 mg/kg body weight of the subject, is about 0.025 mg/kg to about 1.0 mg/kg body weight of the subject, or is about 0.025 mg/kg to about 0.5 mg/kg body weight of the subject.

17. The method of claim 16, wherein the therapeutically effective dose of the opioid receptor mu antagonist is about 0.1 mg/kg to about 0.2 mg/kg body weight of the subject; and the therapeutically effective dose of the opioid receptor kappa agonist is about 0.05 mg/kg to about 0.4 mg/kg body weight of the subject, or the therapeutically effective dose of the opioid receptor kappa agonist is about 0.025 mg/kg to about 0.1 mg/kg body weight of the subject.

18. The method of claim 12, wherein the therapeutically effective dose of the opioid receptor mu antagonist is the same as the therapeutically effective amount of the opioid receptor kappa agonist.

19. The method of claim 18, wherein the therapeutically effective dose of the opioid receptor mu antagonist and the opioid receptor kappa agonist is about 0.05 mg/kg to about 2.5 mg/kg.

20. The method of claim 12, wherein the therapeutically effective dose of the opioid receptor mu antagonist is at a ratio of about 4:1 to about 1:8 relative to the therapeutically effective amount of the opioid receptor kappa agonist.

21. The method of claim 20, wherein the therapeutically effective dose of the opioid receptor mu antagonist is at a ratio of about 4:1, about 1:4, about 1:5, or about 1:8 relative to the therapeutically effective amount of the opioid receptor kappa agonist.

22. The method of claim 21, wherein the wherein the therapeutically effective dose of the opioid receptor mu antagonist is about 0.05 mg/kg to about 0.1 mg/kg.

23. The method of claim 12, wherein the composition is administered intranasally.

24. The method of claim 12, wherein the composition reverses an opioid overdose in the subject.

25. The method of claim 24, wherein the composition reverses the opioid overdose in the subject within about three minutes.

26. The method of claim 12, wherein the composition blocks the opioid receptor in the subject by binding to the opioid receptor, by blocking the binding of an opioid to the opioid receptor, or a combination thereof.
